(19) United States
(12) Patent Application Publication
(10) Pub. No.: US 2008/0227819 A1
(43) Pub. Date: Sep. 18, 2008

(54) Method for identifying the agonistic activity of a target compound on a potassium channel

(75) Inventors: Timo Struenker, Koeln (DE); Markus Cavalar, Aachen (DE); Gregor Bahrenberg, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/047,989

(22) Filed: Mar. 13, 2008

(30) Foreign Application Priority Data
Mar. 13, 2007 (DE) 10 2007 012 029.1

(51) Int. Cl. C12Q 1/02; A61K 31/165; A61K 31/444

(52) U.S. Cl. 514/332; 435/29; 514/617

(57) ABSTRACT

A method for identifying the agonistic activity of a target compound on a potassium channel by a) providing a population of cells expressing a potassium channel and, optionally providing, a protein-based fluorescent-optical voltage sensor, b) if necessary, incubating the cells with a voltage-sensitive fluorescent dye, c) adding the target compound to the reaction batch of a) or b), d) determining a value F₁ of the fluorescence intensity of the cells, e) adding potassium ions in a physiologically acceptable concentration f) determining a value F₂ of the fluorescence intensity of the cells, and g) comparing the fluorescence intensity F₂ with the fluorescence intensity F₁ and determining the agonistic activity of the target compound on a potassium channel therefrom. A potassium channel agonist isolated and purified by this method, pharmaceutical formulations containing the same, and their use for the treatment of a disease in which potassium channels are involved.

### FIELD OF THE INVENTION

The present invention relates to a method and an assay, respectively, for identifying the agonistic activity of a target compound on a potassium ion channel.

### BACKGROUND AND SUMMARY OF THE INVENTION

Ion channels are essential for the function of nerve cells and muscle cells as well as most other somatic cells. Ion channels are involved in almost all physiological processes, for example, in electric impulses, which are the basis of sensoric and motoric functions in the brain, in controlling the contractile activity of the heart, the smooth musculature in skeleton vessels and bowels as well as in the ingestion of nutrients, hormone secretion, cell secretion and cell development.

Ion channels enable ions to pass the hydrophobic lipid bilayer of the cell membrane, which is decisive for the occurrance and conducting of electric signals in living cells. Ion channels can be classified on the basis of their ion selectivity. Some ion channels are only permeable for a certain kind of ions, for example, for sodium ions (Na⁺), for potassium ions (K⁺) or for calcium ions (Ca²⁺). Other channels cannot differentiate at all or can only badly differentiate between different kinds of ions. They are referred to as non-selective channels. The opening or closing of ion channels can be controlled by different mechanisms. Ligand-dependent ion channels are, for example, controlled by the direct binding of intracellular or extracellular ligands. In contrast, voltage-dependent ion channels react to changes in the membrane potential.

The membrane potential of a cell is caused by the electrical gradient and the concentration gradient of different ions and charged particles at both sides of the cell membrane. The distribution of sodium ions and potassium ions in the intracellular and extracellular medium of the cell is of particular importance in this respect. The distribution of ions is based on the selective permeability of the membrane for the different ions and the active transport of ions by means of ion pumps.

The activity of these ATP-powered “pumps” in the cell leads to an uneven distribution of different ions—particularly K⁺, Na⁺, Ca²⁺, Cl⁻ and organic anions—at both sides of the cell membrane and, thus, to ion gradients over the membrane. Therefore, in a resting state there is an excess of negative charges at the inside of the membrane compared to the outside of the membrane. This separation of charges results in a deviation in the electrical potential over the cell membrane, which is referred to as membrane potential (Vₘ).

In a resting state the membrane potential of neurons and muscle cells is in the range of −60 mV to −80 mV (so-called resting membrane potential). Electrical signals in neurons and muscle cells result from short-term changes in the membrane potential. These changes are elicited by a transient opening and closing of ion channels, which leads to a flow of electric current over the membrane. If the separation of charges at the membrane is lowered thereby, it is referred to as a depolarisation of the membrane—the membrane potential becomes less negative. An increase in the separation of charges is referred to as hyperpolarisation, since a membrane potential that is more negative than the resting membrane potential is set up.

The resting membrane potential is a so-called diffusion potential and is determined by various factors: uncharged molecules, such as oxygen, carbon dioxide, urea, etc. can pass the hydrophobic lipid bilayer of the cell membrane without any hindrance. However, ions, charged particles, can only pass the cell membrane by means of selective, protein-based pores (ion channels). Due to their selective permeability for certain ion species a cell membrane equals a semi-permeable membrane. The activity of ATP-powered “pumps” in the membrane of a cell leads to an uneven distribution of different ions—particularly K⁺, Na⁺, Ca²⁺, Cl⁻ and organic anions—at both sides of the cell membrane and, thus, to ion gradients over the membrane. The intracellular and extracellular concentration of the important kinds of ions is depicted in Table 1. The semi-permeability of the cell membrane and the uneven distribution of ions at both sides of the cell membrane result in the development of the resting membrane potential.

TABLE 1
concentration of the most important kinds of ions inside
and outside a cell

| | extracellular | intracellular |
|---|---|---|
| ion | concentration (mM) | concentration (mM) |
| Na⁺ | 145 | 12 |
| K⁺ | 4 | 155 |
| Ca²⁺ | 1.5 | 0.0001 |
| Cl⁻ | 123 | 4.2 |

To clarify this connection, first a cell whose membrane is only permeable for one kind of ions, e.g. for potassium ions (K⁺), is regarded in a simplified manner. K⁺ ions exist in a much higher concentration inside a cell compared to its environment. Therefore, K⁺ ions diffuse from the inside of the cell to the outside of the cell along its chemical gradient. The diffusion of K⁺ out of the cell is, however, self-limited. The outflow of the positively charged K⁺ ions and the resulting excess of impermeable, negatively charged anions within the cell lead to the fact that the inside of the cell becomes negative. Based on this separation of charges an electrical potential difference develops over the cell membrane. The more K⁺ ions leave the cell now, the higher becomes the separation of charges and the higher becomes the potential difference; the inside of the cell becomes continuously more negative. Finally, this electrical potential difference antagonises the further outflow of the positively charged K⁺ ions.

This means that the diffusion of potassium over the cell membrane is influenced by two opposite forces: a) the chemical driving power, which depends on the concentration difference of potassium, and b) the electrical driving power, which is determined by the electrical potential difference over the membrane.

Therefore, after a short time the diffusion of potassium leads to a membrane potential at which the chemical driving power, which conducts the K⁺ ions out of the cell, equals exactly the electrical driving power of the membrane potential, which antagonises the outflow of K⁺ ions. Exactly at the moment when this membrane potential is reached, there is no more net current of K⁺ ions over the membrane. This potential is referred to as so-called “potassium equilibrium potential” (E_{K}).

$$Nernst equation for potassium ( at 37 ° C . ) \\ {E}_{K} = 61 \; 4 mV \times log ( \frac{{[ {K}^{+} ]}_{a}}{{[ {K}^{+} ]}_{i}} ) \\ [ ion ] = concentration , ( a ) extracellular ( i ) intracellular \qquad Equation 1$$

By means of the so-called Nernst equation the equilibrium potential for potassium ions can be calculated.

According to the Nernst equation (Equation 1) the potassium equilibrium potential is determined by the quotient of the potassium concentrations at both sides of the cell membrane. Using the potassium concentrations indicated in Table 1, there is a potassium equilibrium potential of −98 mV. The membrane potential of a cell whose membrane is selectively permeable for potassium would, thus, exactly correspond to E_{K} and be −98 mV.

TABLE 2
extracellular and intracellular concentrations of the
most important kinds of ions (cf. Table 1) and equilibrium
potential resulting therefrom

| | extracellular | intracellular | |
|---|---|---|---|
| | concentration | concentration | equilibrium |
| ions | (mM) | (mM) | potential (mV) |
| Na⁺ | 145 | 12 | +67 |
| K⁺ | 4 | 155 | −98 |
| Ca²⁺ | 1.5 | 0.0001 | +129 |
| Cl⁻ | 123 | 4.2 | −90 |

Usually, there are not only ion channels conducting potassium in the membrane of a cell but also channels for other species of ions. As depicted in Table 1 and Table 2, there is also a concentration gradient for these ions over the cell membrane. Consequently, it is also possible to determine a certain equilibrium potential for these ions. Table 2 shows the equilibrium potentials of the most important ions. E.g., the gradient for sodium ions is exactly inverse to the gradient of potassium ions. Consequently, the equilibrium potential for sodium is far in the positive range with +67 mV.

The way in which the different ion gradients contribute to the resting membrane potential of a cell that is permeable for potassium ions, sodium ions and chloride ions is described by the so-called Goldman-Hodgkin-Katz voltage equation (Equation 2).

$$Goldman \text{-} Hodgkin \text{-} Katz voltage equation ( 37 ° C . ) \\ {V}_{m} = 61 \; 4 mV \times log \frac{{{P}_{K} [ {K}^{+} ]}_{a} + {{P}_{Na} [ {Na}^{+} ]}_{a} + {{P}_{Cl} [ {Cl}^{-} ]}_{i}}{{{P}_{K} [ {K}^{+} ]}_{i} + {{P}_{Na} [ {Na}^{+} ]}_{i} + {{P}_{Cl} [ {Cl}^{-} ]}_{a}} \\ [ ion ] = concentration , ( a ) extracellular ( i ) intracellular . Px = permeability of the membrane for ion X . {V}_{m} = membrane potential \qquad Equation 2$$

It can be deduced from Equation 2 that the membrane potential of a cell is not only determined by the quotients of the ion concentrations at both sides of the cell membrane but most of all by the permeability (P) of the membrane for the respective ion. The higher the permeability (often referred to as conductivity) of the membrane for a certain ion, the higher the contribution of this ion to the membrane potential. That is, the membrane potential represents in a certain way the weighted mean value of the equilibrium potentials for the different ions.

The permeability of the membrane for the respective kind of ions is solely determined by the number and activity of the respective ion channels that conduct this ion. In a resting state there are only very few opened sodium channels, chloride channels or calcium channels in the cell membrane. The membrane potential of most cells in a resting state is mainly determined by a potassium conductivity of the membrane (P_{K}:P_{Cl}:P_{Na}=1:0.45:0.04). Therefore, neurons and muscle cells have a resting membrane potential of −60 to −80 mV (see above), which is close to the equilibrium potential for potassium.

A change in the membrane potential of a cell takes place if the permeability of the membrane for one kind of ions changes. E.g., an activation of sodium channels shifts the membrane potential in the direction of the equilibrium potential for sodium—the membrane potential becomes more positive.

The membrane potential can also adopt a new value if the intracellular or extracellular concentration of an ion changes (cf. Equation 2). In this case the extent of the change in the membrane potential depends on the extent to which the membrane potential as a whole is determined by this ion. Thus, changes in the extracellular sodium concentration, chloride concentration or calcium concentration have only minor influence on the membrane potential. The membrane potential is, however, clearly influenced by the extracellular potassium concentration.

The human genome comprises about 50 different potassium channel genes. Potassium channels can be found in almost any cell type of an organism and represent the biggest and most diverse family of ion channels. The activity is modulated by different physiological stimuli depending on the type of channel, e.g., changes in the membrane potential (voltage-dependent channels), G proteins, calcium ions, nucleotides (ATP) etc. In most cells the resting membrane potential is determined by potassium channels (see above). Potassium channels decisively influence the frequency and the time response of action potentials as well as their conducting in neurons and muscle cells. Furthermore, they regulate the electrical excitability of these cells and they play a decisive role in some secretory processes, such as the secretion of insulin.

Active agents may influence the activity of potassium channels in different ways. Channel blockers usually block the channel pore from the intracellular and extracellular side of the membrane.

Moreover, the interaction of a channel blocker with an accessory subunit may, e.g., result in the fact that the channel pore closes and/or cannot be opened by physiological stimuli (e.g. K_{ATP} channel blocker). By an interaction with accessory subunits channel openers may also lead to an opening of the pore (e.g. K_{ATP} channel opener).

However, most potassium channel openers that have a effect on voltage-dependent channels act as gating modifier. The substances influence the voltage dependency of the activation of the channels. Due to the binding of the substance, the activation curve of the channels shifts to the more negative potentials. Consequently, at the same membrane potential more channels are opened. Retigabine, a known KCNQ channel opener, acts, e.g., via this mechanism.

It seems that the pharmacological manipulation of certain potassium channels is of therapeutic avail for a variety of diseases and is even accepted as an effective therapy for some diseases. Potassium channels are, therefore, important drug targets. The fields of indication are, among other fields, neurological diseases, such as epilepsies (e.g. KCNQ channels), but also high blood pressure (e.g. K_{ATP} channels, K_{Ca} channels), cardiac arrhythmias (e.g. K_{ATP} channels, hERG channels, KCNQ channels, HCN channels), conditions of pain (e.g. KCNQ channels), diabetes (e.g. K_{ATP} channels), asthma, incontinence (e.g. K_{ATP} channels) and other diseases. Consequently, providing compounds that activate or inactivate ion channels includes a potential to develop new and highly effective therapeutic concepts.

In the last few years a wide range of new ion channels and/or subunits of ion channels has been identified. This has increased the interest in new active agents or drugs, respectively, that change the activity of such channels.

Searching for new potent active agents that influence potassium channels, requires the development of assays allowing to test the influence of substances on the activity of the respective type of potassium channel with a throughput as high as possible (review: FORD ET AL., Progress in Drug Research, vol. 52, p. 133 et seq., 2002).

At present, various techniques are available for measuring the activity of potassium channels: The so-called patch-clamp technique is regarded as gold standard for examining the activity of all kinds of ion channels.

For a long time this technique was regarded as inappropriate for screening active agents due to the very low throughput. To make the patch-clamp technique useable as HTS technology (high throughput screening), the automation of this method is intensively promoted. First promising assays, e.g. for hERG potassium channels (human ether-a-go-go-related gene), have already been developed by means of these systems. This technology is particularly auspicious for assays with voltage-activated potassium channels, such as KCNQ channels. This technology allows to test the effect of substances on the channels under physiological conditions. However, currently, non-invasive, cellular assays still allow for a faster search for active agents.

Potassium channels always conduct a certain degree of rubidium (Rb⁺) besides potassium, too. Rubidium ions and potassium ions have very similar characteristics, and, therefore, it is difficult for potassium channels to discriminate between these two ions. Under physiological circumstances Rb⁺ is not of importance, since it only occurs in traces in the body. The conductibility of the channels for rubidium was, however, used for developing non-invasive assays. Classic Rb⁺ flow assays in the microtiter plate format were developed for potassium channels, such as the voltage-dependent KCNQ- hERG-, K_{V}1.3 channels and the K_{ATP} channels (e.g. SCOTT ET AL., Analytical Biochemistry, 319 (2003), 251-257). First, the cells expressing the respective channel are loaded with radioactive rubidium. Then, e.g., the efflux of radioactive rubidium from the culture cells after a targeted activation of the respective potassium channel is measured. Today it is also possible to use non-radioactive rubidium for such assays. By means of the atom absorption spectrometry the rubidium efflux will then be demonstrated. However, the experimental work and the costs for such assays are very high and, thus, these methods have not been able to prevail over other methods. Not only the patch-clamp technique but also the rubidium flux assays are principally suitable for searching for potassium channel openers and blockers.

However, most widely spread are cell-based assay technologies, in which the effect of substances on the potassium channels is optically read by means of special fluorescent dyes (e.g. WHITEAKER ET AL., Journal of Biomolecular Screening, vol. 6, no. 5, 2001). So-called voltage-sensitive fluorescent dyes are used for this method. These dyes show an intensive fluorescence in the cell and in the cell membrane, whereas they fluoresce only very weakly in the aqueous extracellular phase. Regarding these dyes, a change in the membrane potential leads to a redistribution of dye molecules between the extracellular phase, the cell membrane and the inside of the cell. Thus, a change in the membrane potential leads to a change in the fluorescence intensity of the cell. In this case a depolarisation of the membrane potential leads to an increase in the fluorescence intensity, whereas the fluorescence intensity decreases if the cell hyperpolarises. The absolute change in the fluorescence intensity can amount to about 1-2.5% per 1 mV change in the membrane potential, depending on the dye and the cell system.

Hereinafter, it will be shown how to search, by means of voltage-sensitive fluorescent dyes, for active agents influencing the activity of potassium channels.

“Direct” membrane potential assays for searching for potassium channel modulators:

It is possible to identify potassium channel blockers (depolarisation assay) as well as potassium channel openers (hyperpolarisation assay) with this type of assays. The influence of active agents on the membrane potential of cells expressing the respective potassium channel is directly monitored. First, the cells are incubated with a fluorescent dye, and then the fluorescence intensity of the cells is recorded before and after the application of the active agent.

Additional advantages and novel features of the present invention will be set forth in part in the description which follows and in part will become apparent to those skilled in the art upon examination of the following or may be appreciated further by practice of the invention.

### BRIEF DESCRIPTION OF THE DRAWINGS

Features and advantages of the present invention will become apparent to those skilled in the art from the following description with reference to the drawings, in which:

FIG. 1 shows fluorescent intensity over time after application of a KCNQ channel blocker (XE991);

FIG. 2 shows fluorescent intensity over time after application of a KCNQ channel opener (retigabine);

FIG. 3 shows fluorescent intensity over time after providing potassium in the presence and absence of a KCNQ channel blocker (XE991);

FIGS. 4 and 4B show the influence of retigabine on fluorescent intensity after providing potassium;

FIGS. 5A and 5B show the influence of retigabine on fluorescent intensity after providing potassium;

FIGS. 6A and 6B show the influence of substance A on fluorescent intensity after providing potassium; and

FIGS. 7A and 7B show the influence of substance B on fluorescent intensity after providing potassium.

### DETAILED DESCRIPTION

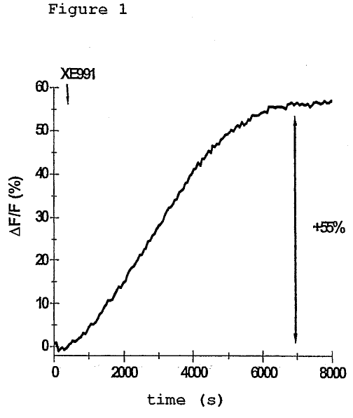

FIG. 1 exemplifies the time response of the fluorescence intensity of cells expressing KCNQ channels before and after the application of a saturating concentration of XE991, a known KCNQ channel blocker. After the application of the KCNQ channel blocker an increase in the fluorescence intensity by about 55% is observed. By blocking the KCNQ channels with XE991, the permeability of the membrane for potassium ions decreases. According to the aforementioned Equation 2, the influence of the potassium equilibrium potential on the membrane potential of the cells is, therefore, reduced, and a new, more positive membrane potential develops. This depolarisation leads to the increase in the fluorescence intensity of the cells.

Depending on the type of potassium channel and the cell system, an increase by 100% and more in the fluorescence intensity by application of a potassium channel blocker can be observed in such “depolarisation assays”. In voltage-dependent potassium channels, such as the KCNQ channels, the changes in the fluorescence potential and the membrane potential are, however, rather low.

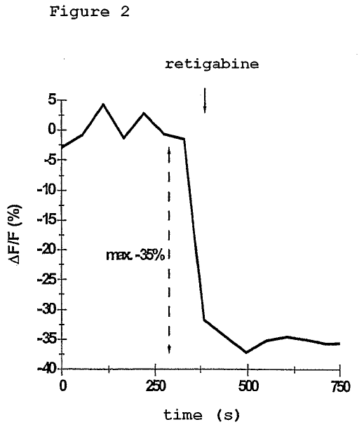

Active agents that open potassium channels in the cell membrane (and shift their voltage-dependence of the activation to more negative potentials, respectively; so-called gating modifiers) also lead to a change in the membrane potential. FIG. 2 exemplifies the time response of the fluorescence intensity of cells expressing KCNQ channels before and after the application of retigabine, a known KCNQ channel opener (and gating modifiers, respectively). The application of retigabine results in a decrease in the fluorescence intensity of about 35-40%. Opening KCNQ channels by retigabine, increases the potassium permeability of the membrane. According to the aforementioned Equation 2, the influence of the potassium equilibrium potential on the membrane potential of the cells is thus increased, and a new, more negative membrane potential develops. This hyperpolarisation leads to a decrease in the fluorescence intensity of the cells.

The dynamic range of such hyperpolarisation assays for searching for potassium channel openers (and gating modifiers, respectively) is, however, not higher than 50% and, thus, rather small. The main reason for this is that the extent of the hyperpolarisation, which may be caused by potassium channel openers, is limited. At most, the membrane potential may adopt the value of the potassium equilibrium potential, and, thus, the degree of the hyperpolarisation is mostly limited to 20-30 mV.

Besides the rather small dynamic range not only the depolarisation assays for potassium channel blocker but also the hyperpolarisation assays for potassium channel openers (or gating modifiers) have other disadvantages:

The fluorescent dyes used tend to interact with the test substances. This leads often to non-specific changes in the fluorescence of the dyes of up to 20% and more. It is often difficult to differentiate between these non-specific changes in the fluorescence and the channel-specific signals, which might lead to an increased number of false negative and false positive results in the high pressure screening.

Therefore, an alternative “indirect” assay method, so-called ion jump assays, is mostly used for searching for potassium channel blockers.

In this kind of membrane potential assays, which have exclusively been used for searching for potassium channel blockers so far, the effect of substances on the respective potassium channels is indirectly determined. Therefore, the cells expressing the respective potassium channel are incubated with a voltage-sensitive fluorescent dye first. In a second step the active agents are applied. Then the fluorescence intensity of the cells before and after an application of potassium ions to the extracellular medium will be monitored. The extracellular potassium ion concentration is increased from 1-5 mM to 50-100 mM by the K⁺ application.

The increase in the extracellular potassium concentration results in a more positive equilibrium potential for potassium (Equation 1). Since the membrane potential of the cells is also determined by the equilibrium potential for potassium, the cells depolarise due to the K⁺ application. However, the degree of the depolarisation depends on the degree of permeability of the membrane for potassium (Equation 2). The lower the permeability for potassium, the less the depolarisation and the less the change in the fluorescence of the cells due to the K⁺ application. The permeability of the membrane for potassium decreases if the potassium channels are blocked. Consequently, in the presence of a blocker the increase in the fluorescence of the cells is less after a K⁺ application compared to the increase in the fluorescence in the absence of a blocker.

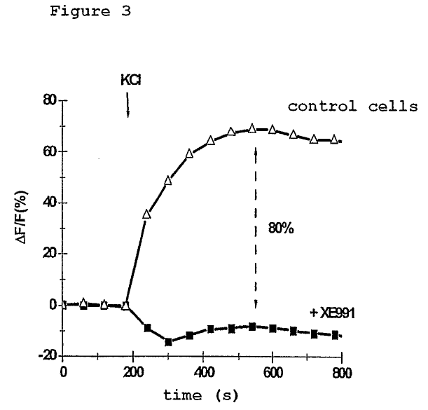

FIG. 3 exemplifies the fluorescence response of cells expressing KCNQ channels to the injection of potassium to the extracellular medium in the presence and in the absence of a KCNQ channel blocker. In the absence of a KCNQ channel blocker (control cells) the potassium application leads to an increase in the fluorescence intensity by about 80%. By contrast, there is no increase in the fluorescence after the potassium application if there is a saturating concentration of the known KCNQ channel blocker XE991. The difference of the amplitudes of the fluorescence responses in the presence and in the absence of a KCNQ channel blocker, the so-called dynamic range of the assay, is about 80% in this case.

Depending on the type of potassium channel and the cell system, the amplitudes of the K⁺-induced changes in the fluorescence in the presence and absence of a blocker can differ by up to 300% in such ion jump assays. Because of this usually large dynamic range, the large fluorescence signals and the good reproducibility, ion jump assays have been widely applied for searching for potassium channel blockers for many years (e.g. FALCONER ET AL., Journal of Biomolecular Screening, vol. 7, no. 5, 2002; WOLFF ET AL., Journal of Biomolecular Screening 8(5), 2003). So far they have not been used for searching for potassium channel openers. Up to now nothing has been known about the effect of potassium channel opener in ion jump assays either.

The aforementioned ion jump assays are robust and established assays for detecting potassium channel blockers, i.e. compounds that block or close potassium channels.

By contrast, there are only the hardly satisfying and not reliable hyperpolarisation assays for detecting compounds that open ion channels and in particular potassium channels (so-called channel opener or also agonists or also gating modifier). As opposed to the above-described robust and established ion jump blocker assays, the membrane potential assays known so far for searching for potassium channel openers, in particular voltage-dependent potassium channels, such as KCNQ channels, are clearly limited. The dynamic range, the reliability and the reproducibility are usually low. For searching for channel openers only hyperpolarisation assays have been used so far (e.g. WHITEAKER ET AL., Journal of Biomolecular Screening, vol. 6, No. 5, 2001; GOPALAKRISHNAN ET AL., The Journal of Pharmacology and Experimental Therapeutics, vol. 303, no. 1 (2002), pp. 379-386, and British Journal of Pharmacology (2004), 143, 81-90). These assays provide only very small measurement windows (small dynamic range), in particular regarding voltage-dependent potassium channels. The small measurement window is caused by the biophysical characteristics of the channels: culture cells, such as CHO cells or HEK cells, usually have a membrane potential of about −20 to −40 mV; far away from the potassium equilibrium potential. The reason for this is that these cells hardly express own potassium conductivities. Consequently, the membrane potential of these cells is hardly dominated by the potassium equilibrium potential.

Due to the heterologous expression of a voltage-dependent potassium channel the membrane potential of the culture cells changes. Because of the basal activity of the voltage-dependent potassium channels the membrane potential shifts in the direction of the potassium equilibrium potential—the membrane potential becomes more negative. However, this hyperpolarisation is self-limiting, since the channels themselves are closed by the hyperpolarisation. Consequently, there is a new state of equilibrium. In CHO cells and HEK cells the expression of a voltage-dependent potassium channel results in a membrane potential of about −60 to −70 mV.

If the channels are now activated by a potassium channel opener, the membrane potential continues shifting in the direction of the potassium equilibrium potential, which is at about −90 mV. Thus, the membrane potential cannot be more negative than the potassium equilibrium potential. Since the cells already have a membrane potential of about −60 to −70 mV due to the basal activity of the potassium channels, the amplitude of the channel-opener-induced hyperpolarisation is limited to 20 to 30 mV.

Since exactly this channel-opener-induced hyperpolarisation has been directly followed by means of voltage-sensitive fluorescent dyes in the hyperpolarisation assays for voltage-sensitive potassium channels used so far, the amplitude of the fluorescence responses is correspondingly limited, too.

Therefore, the dynamic range of the hyperpolarisation assays used so far for voltage-dependent potassium channels is small, which has complicated a reliable analysis of structure and effect up to now.

Regarding the non-voltage-dependent channels, such as K_{ATP} channels or also some calcium-activated potassium channels, the dynamic range of the hyperpolarisation assays is often slightly larger, since these channels have a low basal activity in the absence of a channel opener and, thus, the membrane potential of the cells is more positive in the absence of a channel opener.

The small dynamic range of the hyperpolarisation assays for potassium channel openers and also the frequent non-specific interactions between the dye and the active agent may prevent a reliable analysis of structure and effect and lead to rather unsatisfying results, respectively. Thus, there is a need for assays with potassium channel openers, in particular openers of voltage-dependent potassium channels.

Therefore, it is the objective of the present invention to provide a method for detecting potassium channel openers easily and reliably. The method shall be characterized in that it is fast, efficient and highly sensitive and shall be universally suitable for testing any potassium channels on compounds opening them. Furthermore, the method shall be suitable for testing the ability of any compound to open a potassium channel. Moreover, the method shall have a low false positive recall ratio, shall lead to few false negative results and shall be characterized in that it is highly reproducible. The method shall also be suitable for bench-scale use and for a high throughput screening (HTS) as well as for automation. In addition, the method shall be cost-effective and shall not lead to unnecessary environmental pollution, e.g. by radioactive or toxic chemicals. Preferably, the method shall be suitable for searching for openers of voltage-dependent potassium channels.

Surprisingly, it has now been found that the measuring principle of ion jump assays can be used successfully and very sensitively for identifying compounds that open potassium channels and gating modifiers, respectively, which influence the voltage-dependency of voltage-dependent potassium channels. A gating modifier acts as a potassium channel opener if the voltage-dependency of the potassium channels and the semi-maximal activating voltage, respectively, is shifted to more negative potentials. It was also found that by modifying the ion jump measuring principle, a new assay could be established for simply and effectively determining the agonistic, i.e. the opening and, respectively, the activation voltage dependency modifying activity of a target compound on a potassium channel. It was found that this assay format with a high sensitivity is appropriate to specifically identify and characterize potassium channel openers. It was also found out that the measuring principle of ion jump assays is particularly appropriate to identify compounds that open voltage-dependent potassium channels.

Therefore, the present invention relates to a method for identifying the agonistic activity of a target compound on a potassium channel characterized in that a) a population of cells expressing a potassium channel and, optionally, a protein-based fluorescent-optical voltage sensor is provided, b) optionally, the cells according to a) are incubated with a voltage-sensitive fluorescent dye, c) the target compound is added to the reaction batch of a) or b), d) a value F₁ of the fluorescence intensity of the cells is determined, e) potassium ions in a physiologically acceptable concentration are added, f) a value F₂ of the fluorescence intensity of the cells is determined, and g) the fluorescence intensity F₂ is compared with the fluorescence intensity F₁ and the agonistic activity of the target compound on the potassium channel is determined therefrom. Thus, F1 and F2 are preferably used in calculation as follows:

$$( \frac{{F}_{2} - {F}_{1}}{{F}_{1}} ) \times 100 = \frac{Δ F}{F} ( \% )$$

To determine if a substance has agonistic activity,

$$\frac{Δ F}{F}$$

can, e.g., be compared with

$${( \frac{Δ F}{F} )}_{K}$$

by control cells.

$${( \frac{Δ F}{F} )}_{K}$$

is determined by a) incubating a population of cells expressing the potassium channel with a fluorescent dye, b) adding only a simple buffer solution instead of a target compound to the reaction batch of a), c) determining a value F_{1K} of the fluorescence activity of the control cells, d) adding potassium ions in a physiologically acceptable concentration, e) determining a value F_{2K} of the fluorescence activity of the control cells, and f) preferably using F_{2K} and F_{1K} in calculation as follows:

$$( \frac{{F}_{2 K} - {F}_{1 K}}{{F}_{1 K}} ) \times 100 = {( \frac{Δ F}{F} )}_{K} ( \% )$$

A substance has agonistic activity on the potassium channel if

$$\frac{Δ F}{F}$$

is larger than

$${( \frac{Δ F}{F} )}_{K} \text{:}$$

$$\frac{Δ F}{F} 〉 {( \frac{Δ F}{F} )}_{K}$$

Independent of the comparison of

$$\frac{Δ F}{F} with {( \frac{Δ F}{F} )}_{K} ,$$

agonistic activity of a target compound may be deduced if an increase in

$$\frac{Δ F}{F}$$

can be observed with an increasing dosing of the target compound.

Agonistic activity of a target compound on a potassium channel means any activity of the target compound opening this channel. There is an opening activity if the voltage-dependency of the channels and/or the semi-maximal activation voltage is shifted to more negative potentials. Thus, this term also includes the so-called gating modifiers. By means of the method according to the present invention all types of potassium-channel-opening compounds can be detected, independent of whether or not these compounds open the channel completely or only to a certain extent.

It is surprising that this assay principle, which has been unknown for ion channel openers and in particular potassium channel openers so far, is very superior to the known hyperpolarisation assays. The dynamic range of this new ion jump assay is 250% for KCNQ channels and is, thus, six times larger than the dynamic range of a hyperpolarisation assay, which is only about 35-40% for KCNQ channels.

This innovative assay for potassium channel openers is extremely reliable, reproducible and allows for a reliable structure-effect-analysis. The assay format for potassium channel openers according to the present invention is, therefore, superior to the hyperpolarisation assay formats used so far.

The assay according to the present invention has also the advantage that the ion jump principle does not follow the limited channel-opener-induced hyperpolarisation. In ion jump assays the activity of the potassium channels is indirectly determined via the potassium-dependency of the membrane potential. The limitations of the hyperpolarisation assays used so far are not of importance here. Therefore, the dynamic range of the new ion jump assay format is much larger than the dynamic range of usual assays, which allows for a more reliable and more sensitive search for active agents, in particular for openers of voltage-sensitive potassium channels. The newly developed assay is, therefore, superior to the assay formats used so far, particularly for searching for openers of voltage-dependent potassium channels.

Furthermore, the very large dynamic range of the new assay method allows to identify potassium channel opener, in particular openers of voltage-sensitive potassium channels, which have only a weak agonistic effect on the respective potassium channels. It is difficult to identify such active agents with the hyperpolarisation assays used so far, since the dynamic range of such assays is usually very small.

Since the effect of substances on the potassium channels is also read out indirectly by means of the potassium-induced depolarisation in the new assay method for searching for potassium channel openers, non-specific interactions between the dye and the active agent can be neglected, whereby the principle and the advantages of the voltage-sensitive dyes can basically be utilised. Compared to the conventional hyperpolarisation assays, this clearly reduces the occurrence of false positive and false negative results and leads to a higher sensitivity of the assay format according to the present invention.

In the method according to the present invention method steps that are already known from ion jump assays are used in a way known per se. The modifications of the ion jump assay set forth below can preferably been carried out to improve and, respectively, increase the suitability and sensitivity of the method according to the present invention for identifying compounds that open a potassium channel.

According to the present invention first a population of cells expressing the potassium channel is incubated with a voltage-sensitive fluorescent dye. Any cell populations can be used as long as they express the respective ion channel to be tested. Such cell lines are well known to the person skilled in the art. Examples of them are transiently transfected cell lines, stable cell lines, primary cell cultures and tissue cells as well as cell lines expressing a potassium channel endogenously. Examples of the latter are F-11 cells or PC 12 cells, which endogenously express KCNQ channels.

The cells are grown in a way known per se in known culture media. The cells are grown in a way that is known in the state of the art for the cells expressing the respective potassium channel. For example, CHO cells (Chinese hamster ovary cells) expressing KCNQ channels are cultivated in rolling bottles as suspension culture or adherently in Petri dishes, preferably in cell culture bottles. A Minimum Essential Medium (MEM) with an addition of Fetal Calf Serum is preferably used as a culture medium. Most preferably, the cells are cultivated in a Minimum Essential Medium (MEM), a medium 22571 1×liquid (Invitrogen) with 10% Fetal Calf Serum (FCS) (Gibco, heat inactivated) at 37° C., 5% CO₂ and 95% air humidity. The cells are mechanically or enzymatically detached for the passaging, preferably enzymatically by trypsin, most preferred enzymatically by accutase (company PAA). The cells are transferred in such a rhythm that an approximately 70% confluency state is reached when sowing them on measurement plates or, preferably, according to a determined split regime.

Subsequently, the cells are seeded into measurement plates. To harvest the cells from the cell culture bottles, the cells are washed with an appropriate buffer and mechanically or enzymatically detached from the carrier material. An enzymatic/chemical method with trypsin and ethylenediaminetetraacetic acid (EDTA) is preferably used. It is particularly appropriate to wash the cells at a 70-90% confluency with a buffer of a composition of 0.8-2 mM CaCl₂, 1-5 mM KCl, 1-2 mM KH₂PO₄, 0.2-1 mM MgCl₂, 100-200 mM NaCl, 5-10 mM Na₂HPO₄, 0-10 mM D-glucose and 0-0.5 mM sodium pyruvate buffer and to incubate the cells for 2 to 15 min at 37° C. with a low concentration of trypsin to solubilise them. It is particularly appropriate to wash the cells at 80% confluency with a buffer of a composition of 0.9 mM CaCl₂, 2.7 mM KCl, 1.5 mM KH₂PO₄, 0.5 mM MgCl₂, 140 mM NaCl and 8 mM Na₂HPO₄ and to incubate the cells for 15 min at 37° C. with 2 ml accutase (PAA) to solubilise them.

The solubilised cells are re-suspended in an appropriate amount of the nutrient medium, preferably between 5 and 10 ml. The number of cells is determined in a counting chamber or in a cell counter, preferably in a CASY cell counter (Schärfe System). They are seeded on 1536-well measurement plates, 384-well measurement plates or 96-well measurement plates suitable for measuring fluorescence, preferably on plates whose plastic allows for a better adherence of the cells or on plates that are especially coated for this purpose. 96-well plates with poly-D-lysine coating, poly-L-lysine coating, collagen coating or fibronectin coating are preferably used. Particularly preferred are Corning® CellBIND® 96-well plates (black with clear bottom). Between 10,000 and 40,000 cells per cavity are seeded in the 96-well plates. It is preferred to seed 15,000 to 25,000 cells per well. It is particularly preferred to seed 20,000 cells in 100 μl culture medium and to incubate for 16-32 h, preferably 24 h at 37° C., 5% CO₂ and 95% air humidity. It is especially advantageous to incubate the cells at room temperature for 1 h prior to the incubation at 37° C. to ensure an even adherence of the cells.

Other cell culture protocols may be suitable for other cells expressing potassium channels; however, they are known to the person skilled in the art.

Alternatively, it is also possible to use cells expressing a potassium channel and a protein-based fluorescent-optical voltage sensor. Such cells are well known to the person skilled in the art and have the advantage that the step of the incubation with a voltage-sensitive fluorescent dye can be omitted. The processing of these cells for the method according to the present invention may, e.g., be carried out by following the above-described protocol for cells expressing a potassium channel.

Subsequently, the cells are optionally incubated and, respectively, loaded with a voltage-sensitive fluorescent dye.

Various voltage-sensitive fluorescent dyes for incubating the cells can be used. Such dyes are well known to the person skilled in the art and are exemplified below.

Principally, voltage-sensitive fluorescent dyes can be subdivided into two classes: “slow” and “fast” dyes. “Slow” voltage-sensitive dyes include, e.g., positively loaded dyes on cynanine basis, carbocyanin basis and rhodamine basis, respectively, or also anionic oxonol dyes, bisoxonol dyes or merocyanine dyes [e.g. Membrane Potential Kit, DiSBAC₂(3), DiBAC4 (3), DiOC₂ (3); Dis C₃ (3) Dis C₃ (5)]. The product Membrane Potential Kit is a commercial product of the company Molecular Devices Corporation. This kit is currently offered in two variants:

variant blue: Membrane Potential Assay Kit Blue (catalog nos. R8034 and R8042)
variant red: Membrane Potential Assay Kit Red (catalog nos. R8123 and R8126)

These dyes from Molecular Devices Corporation's Membrane Potential Kit are described in U.S. Pat. No. 6,852,504, the substance of which is specifically incorporated by reference herein.

In literature this kit is also referred to as “FLIPR Membrane Potential Dye”. Both variants of the kit are appropriate to carry out the assay method according to the present invention.

These dyes show an intensive fluorescence in the cell and in the cell membrane, whereas they fluoresce only weakly in the aqueous extracellular phase. In this so-called Nernst dyes a change in the membrane voltage (Vₘ) leads to a redistribution of the molecules of the dye between the extracellular phase, the cell membrane and the inside of the cell. Therefore, a voltage-dependent change in the fluorescence intensity of the cell can be observed. Since the redistribution process is relatively slow, the time constants for the changes in the fluorescence intensity amount to about 1-20 seconds depending on the dye. The absolute change in the fluorescence intensity in this group of dyes is about 1-2.5% per 1 mV change in the membrane potential.

The class of the “fast” voltage-sensitive dyes includes, e.g., styryl dyes and also some oxonols (e.g. di-8-ANEPPS, di-4-ANEPPS, RH-421, RH-237 etc.) and also hemicyanines (e.g. annine-3 and annine-6). These amphiphatic molecules hardly fluoresce in aqueous solution. However, they store in the membrane of cells and show a high fluorescence there.

Regarding the so-called electrochromic dyes (e.g. styryl dyes), a change in Vₘ leads to a change in the distribution of electrons in the dye molecule (the so-called stark shift). Thus, the fluorescent spectrum of the dyes shifts. Other “fast” dyes (e.g. some oxonols) change voltage-dependently their position in the membrane, and this results also in a change in the spectral characteristics. The time constants for these processes amount to less than a millisecond. The voltage-dependent shift of the spectrum is very minor.

Therefore, it is merely possible to detect a minor (approx. 0.1% per 1 mV) change in the fluorescence intensity even if an optimal filter is chosen.

Moreover, according to the invention innovative combinations of dyes, in which a voltage-dependent Förster resonance energy transfer (FRET) takes place, are also interesting for HTS assays. For some time such dye systems for detecting the membrane potential have been available from the companies Invitrogen and Axiom. Since these FRET systems are based on “slow” dyes, the sensitivity is within the order of magnitude of slow dyes.

Besides the dyes described herein, there are also examples of protein-based voltage sensors, e.g. the so-called Flash, Flare, SPARC and VSFP-1 proteins. Such fluorescent protein voltage-sensitive probes (FP voltage sensors) might supersede the dyes used so far in the future. Such voltage sensors are principally suitable for the method according to the present invention. Such protein-based fluorescent-optical voltage sensors are also expressed by the cell expressing the potassium channel.

For the assay system according to the present invention dyes from the group of the “fast” voltage-sensitive dyes are particularly appropriate, such as styryl dyes (ANEPPS dyes), oxonol dyes (e.g. RH421) and hemicyanine dyes (ANINNE dyes). Protein-based voltage sensors, such as Flash, Flare, SPARC and VSFP-1 proteins, with which the cells expressing the respective potassium channel are transfected transiently or in a stable way, are also particularly suitable for the assay format according to the present invention.

For the assay according to the present invention FRET-based combinations of dyes that are based on “slow” dyes, such as those that are, e.g., offered by the companies Invitrogen and Axiom, are preferably used. A FRET-based dye system of the company Invitrogen is commercially available as “voltage sensor probes” (“VSP”). It includes the dyes DisBAC₂(3) or DisBAC₄(3) and the membrane-bound coumarin phospholipid “CC2-DMPE”. The FRET-based dye system of the company Axiom Bioscience is also particularly suitable. It comprises the combination of dyes DisBAC₁(3)/DisBAC₁(5). Other FRET dye systems are also suitable for the assay system.

Dyes from the group of the “slow” voltage-sensitive fluorescent dyes are particularly used, such as Membrane Potential Kit or DiSBAC₂(3) dyes, DiBAC₄(3) dyes, DiOC₂(3) dyes, Dis C₃(3) dyes, Dis C₃(5) dyes.

In the assay format according to the present invention changes in the membrane potential are preferably read out by means of changes in the fluorescence intensity of voltage-sensitive dyes. It is, however, also possible to use a voltage-dependent change in the fluorescence lifetime of dyes or fluorescing proteins as measuring parameters instead of the fluorescence intensity.

A person skilled in the art knows in which way potassium-channel-expressing cells are incubated with a voltage-sensitive fluorescent dye in an appropriate way. Time of incubation, conditions of incubations and the quantitative ratio of cells/fluorescent dye mainly depend on the respective dye used and can be easily detected by a person skilled in the art by routine experiments.

The following descriptions are exemplary for preferred embodiments of the present invention.

For example, to load KCNQ-channel-expressing CHO cells with a voltage-sensitive dye, the culture medium, in which the cells are grown, is removed first.

Then the cells are washed with an appropriate buffer. Subsequently, the cells are covered with the dye dissolved in the buffer and incubated between 2 min and 1.5 h. Thereby, the dye is selected from the above-mentioned dyes. It is preferred to incubate the cells with the dye for 30 to 45 min. Particularly good results may be achieved by incubating the cells for 45 min.

A solution of the composition of 0.8-4 mM CaCl₂, 0-100 mM HEPES, 0.5-10 mM KCl, 0-2 mM KH₂PO₄, 0.4-4 mM MgCl₂, 0-1 mM MgSO₄, 100-200 mM NaCl, 0-40 mM NaHCO₃, 0-10 mM Na₂HPO₄, 0-20 mM D-glucose, 0-0.5 mM phenol red and 0-0.5 mM sodium pyruvate is suitable as a buffer. A buffer of the composition of 1-2 mM CaCl₂, 0.5-6 mM KCl, 0.5-2 mM MgCl₂, 0.2-0.5 mM MgSO₄, 100-150 mM NaCl, 2-5 mM NaHCO₃, 0.2-0.5 mM Na₂HPO₄, 5-10 mM D glucose and 5-15 mM HEPES is particularly suitable.

It is especially advantageous to wash the cells with 200 μl of an extracellular solution (ES; 120 mM NaCl, 1 mM KCl, 10 mM HEPES, 2 mM CaCl₂, 2 mM MgCl₂, 10 mM glucose; pH 7.4) and then to incubate them with 100 μl of a dye solution (1 Vial Membrane Potential Assay Kit, red, dissolved in 200 ml ES solution) for 45 at room temperature.

The above exemplary protocol for the incubation of potassium-channel-expressing cells with a fluorescent dye can be adapted by a person skilled in the art depending on the cells used and the dye used in a known way and, respectively, are determinable by simple routine experiments.

Subsequently, the target compound to be tested is added to the reaction batch. The target compound is added as a solution in an suitable solvent. The amount of the target compound depends on the potassium channel to be tested.

After incubating the dye, the test substance is added in a suitable buffer with a physiologically acceptable proportion of solvent/vehicle and incubated on the cells for about 1 min to 2.5 h. The incubation time depends on the type of the potassium channel to be tested. Preferred incubation times are between 15 min and 1.5 h. It is particularly preferred to incubate the cells with the substance for 30 min. The concentration of the potassium ions added is between 10 mM and 150 mM (final concentration KCl solution), preferably 80 mM to 120 mM, and particularly preferred between 90 mM and 100 mM KCl.

Before adding the potassium ions, a value F₁ of the fluorescence intensity of the cells is determined. The fluorescence intensity is determined in a way known in the state of the art, e.g. with a CCD camera or a photo multiplier.

The measurements can be carried out with any commercially available fluorescence reader; BMG type Fluostar or BMG type Polarstar or Molecular Devices-Flex proved themselves to be especially appropriate. The fluorescence is excited at 525 nm and detected at 560 nm.

Subsequently, the fluorescence intensity of the cells is determined again.

The evaluation of the fluorescence value F₂ takes place at a constant point in time after KCl depolarisation, preferably at the time of the maximum (top value) of the KCl depolarisation. The

$$\frac{Δ F}{F}$$

value of the corresponding test substance is detected by using the F2 value in the substance well and the fluorescence value at a fixed and for all substances identical point in time of the base line of the substance well (F₁):

$$( \frac{{F}_{2} - {F}_{1}}{{F}_{1}} ) \times 100 = \frac{Δ F}{F} ( \% )$$

The average value of the

$${( \frac{Δ F}{F} )}_{K}$$

values of the vehicle controls is subtracted from this substance-specific

$$\frac{Δ F}{F}$$

value and, thus, the substance-specific dynamic range (with the unit percent) is determined. This value may be indicated as percentage of the effect (dynamic range) of a reference substance relevant for the target protein.

A substance has agonistic activity on the potassium channel if

$$\frac{Δ F}{F}$$

is larger than

$${( \frac{Δ F}{F} )}_{K } \text{:} \frac{Δ F}{F} 〉 {( \frac{Δ F}{F} )}_{K}$$

To discriminate real agonistic activity from a normal variance of the measuring values, it is hold that it is only possible to suggest agonistic activity if the following condition is fulfilled:

$$\frac{Δ F}{F} 〉 [ {( \frac{Δ F}{F} )}_{K} + 3 * σ ]$$

$$\frac{Δ F}{F}$$

must be outside of the so-called 3σ interval for the control cells, wherein “σ” is the standard deviation of

$${( \frac{Δ F}{F} )}_{K} .$$

In the case of KCNQ channels it is preferred to determine the fluorescence intensity after 60 s. Depending on the potassium channel and the measuring device, the time of determining the fluorescence intensity can, however, be different. The appropriate point in time can be determined by a person skilled in the art by means of simple routine experiments. A statement on the agonistic activity of the target compound on the respective ion channel can be made on the basis of the quotient of the fluorescence intensity measured before adding the ions for which the ion channel is permeable and after adding these ions.

The method according to the present invention is particularly suitable for a high throughput screening (HTS) for potassium channel openers. The cultivation of cells in 96 well plates, which can be read out by means of respective fluorescence readers, is especially suitable for this method. However, it is also possible to adapt to 384-well plates or 1536-well plates and, thus, another increase in the throughput is possible. Corresponding modifications are known to the person skilled in the art.

The present invention also relates to isolated and purified ion channel agonists that were identified by means of the method according to the present invention. Moreover, the present invention relates to pharmaceutical formulations including the ion channel agonists identified in this way, and to the use of ion channel agonists for preparing pharmaceutical formulations for the treatment of a disease in which ion channels are involved. Corresponding pharmaceutical formulations are formulated by using usual pharmaceutically acceptable excipients. Such formulations are well known to the person skilled in the art. Examples of diseases that can be treated by means of the (potassium) ion channel agonists identified according to the present invention are pain, diabetes, metabolic syndrome, cardiac arrhythmias, epilepsy, high blood pressure, asthma etc.

Ion channel agonists that are already known in the state of the art are excluded from the scope of the present invention.

By means of the assay format according to the present invention highly efficient potassium channel openers can be identified. The high sensitivity of the assay format allows for the identification of compounds that selectively directly affect specific potassium ion channels. The method according to the present invention is basically suitable for examining any potassium channels; however, it is particularly suitable for examining the following potassium channels: voltage-dependent potassium channels of the K_{V} channel family, however, also channels from the family of the inwardly rectifying potassium channels (Kᵢᵣ family), calcium-activated potassium channels (K_{Ca} family) and also two-pore-domain channels, such as TWIK channels, TASK channels and TREK channels (K_{2P} family).

Examples of the aforementioned potassium channels are voltage-dependent potassium channels: K_{V}1.1, K_{V}1.2, K_{V}1.3, K_{V}1.4, K_{V}1.5, K_{V}1.6, K_{V}1.7, K_{V}1.8, K_{V}2.1, K_{V}2.2, K_{V}3.1, K_{V}3.2, K_{V}3.3, K_{V}3.4, K_{V}4.1, K_{V}4.2, K_{V}4.3, K_{V}5.1, K_{V}6.1, K_{V}6.2, K_{V}6.3, K_{V}7.1, K_{V}7.2, K_{V}7.3, K_{V}7.4, K_{V}7.5, K_{V}8.1, K_{V}9.1, K_{V}9.2, K_{V}9.3, K_{V}10.1, K_{V}10.2, K_{V}11.1, K_{V}11.2, K_{V}11.3, K_{V}12.1, K_{V}12.2, K_{V}12.3 calcium-activated potassium channels: K_{Ca}1.1, K_{Ca}2.1, K_{Ca}2.2, K_{Ca}2.3, K_{Ca}3.1, K_{Ca}4.1, K_{Ca}4.2, K_{Ca}5.1

inwardly rectifying potassium channels: Kir1.1, Kir2.1, Kir2.2, Kir2.3, Kir2.4, Kir3.1, Kir3.2, Kir3.3, Kir3.4, Kir4.1, Kir4.2, Kir5.1, Kir6.1, Kir6.2, Kir7.1
two-pore potassium channels: K_{2P}1.1, K_{2P}2.1, K_{2P}4.1, K_{2P}3.1, K_{2P}5.1, K_{2P}6.1, K_{2P}7.1, K_{2P}9.1, K_{2P}10.1, K_{2P}12.1, K_{2P}13.1, K_{2P}15.1, K_{2P}16.1, K_{2P}17.1, K_{2P}18.1

The method according to the present invention is preferably suitable for examining potassium channels of the K_{V}7.x family and the KCNQ family, respectively.

The present invention also relates to the use of an, optionally, modified ion jump membrane potential assay and, respectively, an assay with the aforementioned steps for identifying compounds with agonistic activity on a potassium ion channel, particularly on a voltage-dependent potassium ion channel.

The attached figures explain the present invention in more detail. It is shown in

FIG. 1: Effect of XE991 on the membrane potential of cells expressing KCNQ channels in a depolarisation assay format. The time response of the fluorescence intensity of CHO cells expressing KCNQ channels before and after the application (arrow) of a known KCNQ channel blocker (XE991, 60 μM) is depicted.

FIG. 2: Effect of retigabine on the membrane potential of cells expressing KCNQ channels in a hyperpolarisation assay format. The time response of the fluorescence intensity in CHO cells expressing KCNQ channels before and after the application (arrow) of a known KCNQ channel opener (retigabine, 30 μM) is depicted.

FIG. 3: Influence of XE991 on the fluorescence response after potassium injection to the extracellular medium in a ion jump assay format for searching for potassium channel blockers. The time response of the fluorescence intensity of CHO cells expressing the KCNQ channels before and after the application (arrow) of 100 mM potassium chloride is depicted.

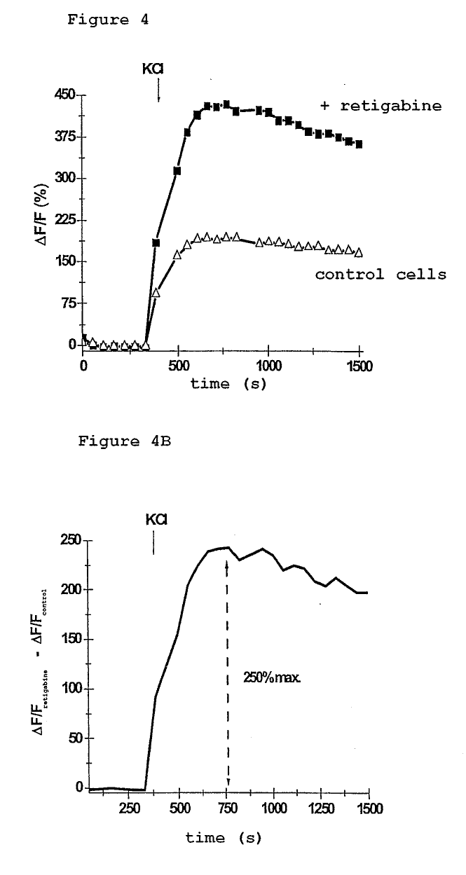

FIG. 4: Influence of retigabine on the fluorescence response after potassium injection to the extracellular medium in an ion jump assay format for detecting potassium channel openers.
A) The time response of the fluorescence intensity of CHO cells expressing the KCNQ channels before and after the application (arrow) of 100 mM potassium chloride is depicted.
B) Difference of the courses of the fluorescence in A.

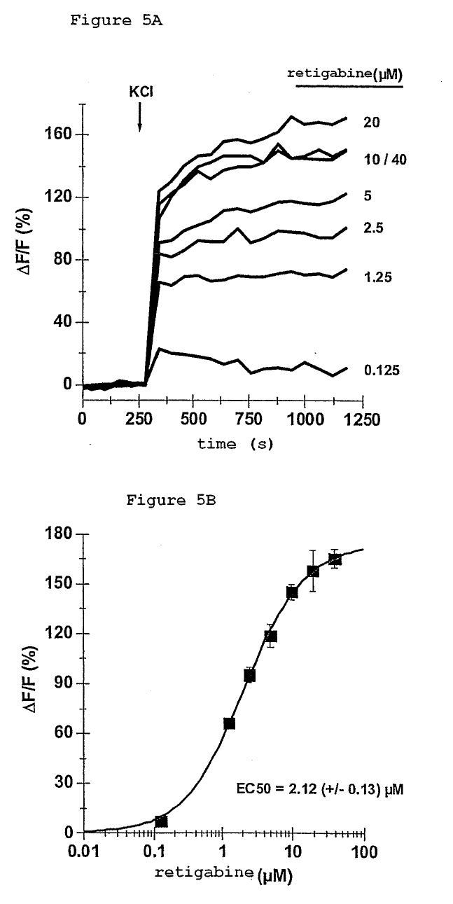

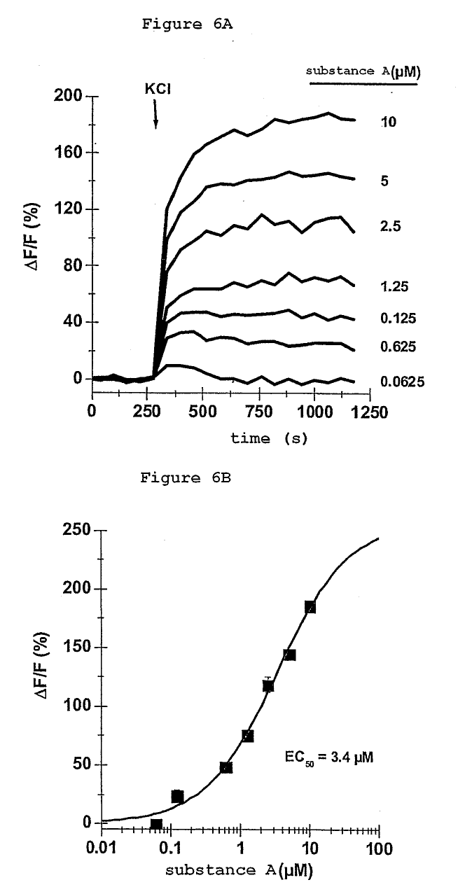

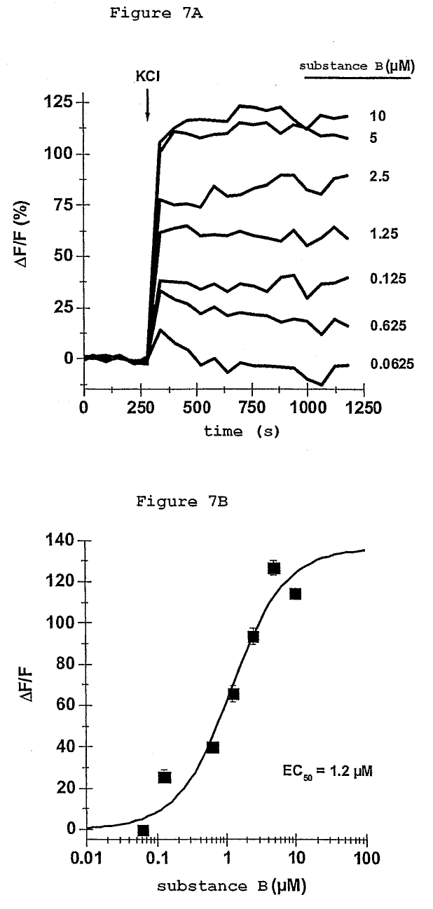

FIGS. 5/6/7: Agonistic activity of retigabine (FIG. 5A+B), substance A (FIG. 6A+B) and substance B (FIG. 7A+B) in the ion jump assay format for detecting potassium channel openers.
A) The time response of the fluorescence intensity of CHO cells expressing KCNQ channels before and after the application (arrow) of 100 mM potassium chloride is depicted. The fluorescence responses of the cells in the presence of different concentrations of retigabine (FIG. 5A), substance A (FIG. 6A) and substance B (FIG. 7A) are shown.
B) The percental changes of the fluorescence intensity (AF/F) after the application of 100 mM potassium chloride are depicted depending on the concentration of retigabine (FIG. 5B), substance A (FIG. 6B) and substance B (FIG. 7B).

The following examples explain the present invention in more detail:

### EXAMPLE 1

### Effect of XE991 on the Membrane Potential of Cells Expressing the KCNQ Channels in a Depolarisation Assay Format

### REFERENCE EXAMPLE

CHO cells (Chinese hamster ovary cells) expressing KCNQ2 channels and KCNQ3 channels were adherently cultivated in a nutrient medium [Minimum Essential Medium (MEM), α medium 22571 1×liquid (Invitrogen), 10% Fetal Calf Serum (FCS) (Gibco, heat inactivated), including selection antibiotics] at 37° C., 5% CO₂ and 95% air humidity in cell culture bottles. At 80% confluency the cells were washed with 1×DPBS without adding Ca²⁺/Mg²⁺ and incubated with 2 ml accutase for 15 min at 37° C. The cells detached from a T75 cell culture bottle were re-suspended in 8 ml nutrient medium. After determining the number of cells in a CASY cell counter, 20,000 cells/well were seeded into Corning® CellBIND® 96-well plates (black with clear bottom) in 100 μl nutrient medium and incubated for 24 h at 37° C., 5% CO₂ and 95% air humidity. Then the cells were loaded with the red FMP dye of the company Molecular Devices. A vial Membrane Potential Assay Kit Red Component A (MPK, bulk-kit) was dissolved in 200 ml extracellular buffer solution (ES, 120 mM NaCl, 1 mM KCl, 10 mM HEPES, 2 mM CaCl₂, 2 mM MgCl₂, 10 mM glucose; pH 7.4). To load the cells with the voltage-sensitive fluorescent dye, the culture medium was discarded first. Then the cells were washed with 200 μl ES buffer; subsequently they were covered with 100 μl of the dye solution and incubated for 45 min. After incubation with the dye, either 50 μl ES (control) or 50 μl of the KCNQ channel blocker XE991 in independent wells in a saturating concentration (final concentration 60 μM) were added and incubated for 2 h13 min with concurrent monitoring of the fluorescence in the reader. The measurements were carried out by a Fluostar or Polarstar fluorescence reader of the company BMG or the FlexStation of the company Molecular Devices. It was excited at a wavelength of 525 nm and detected at a wavelength of 560 nm.

In FIG. 1 the time response of the fluorescence intensity of CHO cells expressing KCNQ channels before and after the application (arrow) of a known KCNQ channel blocker (XE991) is depicted.

The percental change in the fluorescence intensity (ΔF/F) based on the averaged fluorescence intensity before the XE991 application is depicted. The course of the fluorescence of the control cells (application of ES buffer) is subtracted from the XE991-induced fluorescence signals.

### EXAMPLE 2

### Effect of Retigabine on the Membrane Potential of Cells Expressing the KCNQ Channels in a Hyperpolarisation Assay Format

### COMPARATIVE EXAMPLE

The cells were seeded into assay plates and loaded with the dye in exactly the same way as described in Example 1. The measurements were carried out by a Fluostar or Polarstar fluorescence reader of the company BMG or the FlexStation of the company Molecular Devices. It was excited at a wavelength of 525 nm and detected at a wavelength of 560 nm. After incubation with the dye, the base line was monitored for 5 min. Then either 50 up ES buffer (control) or 50 μl of the KCNQ agonist retigabine (30 μM) were added and the fluorescence was monitored in an appropriate reader for 7.5 min.

In FIG. 2 the time response of the fluorescence intensity of CHO cells expressing KCNQ channels before and after the application (arrow) of the known KCNQ channel opener (retigabine, 30 μM) is depicted. The percental change in the fluorescence intensity (ΔF/F) based on the averaged fluorescence intensity before the application of retigabine is depicted. The course of the fluorescence of the control cells (application of buffer) was subtracted from the retigabine-induced fluorescence signal.

### EXAMPLE 3

### Influence of XE991 on the Fluorescence Response After Potassium Injection to the Extracellular Medium in an Ion Jump Assay Format for Searching for Potassium Channel Blockers

The seeding of KCNQ-expressing cells in assay plates, the loading with the dye and the measurement devices to be used as well as the wavelengths to be set are in accordance with Example 1. Either 50 μl ES buffer (control) or 50 μl of the KCNQ blocker XE991 (60 μM) were added into different wells of a plate with KCNQ-expressing cells and incubated for 30 min. Then the base line was monitored in the fluorescence reader for 3-4 min and the fluorescence values were measured for 10 min after the injection of 15 μl of a KCl solution (final concentration 91.8 mM) in each cavity of the plate.

In FIG. 3 the time response of the fluorescence intensity of CHO cells expressing the KCNQ channels before and after the application (arrow) of 91.8 mM potassium chloride is depicted. The fluorescence responses of the cells in the absence (Δ) and presence (▪) of the known KCNQ channel blocker XE991 (60 μM) are shown. The percental change in the fluorescence intensity (ΔF/F) based on the averaged fluorescence intensity before the potassium application is depicted.

### EXAMPLE 4

### Influence of Retigabine on the Fluorescence Response After Potassium Injection to the Extracellular Medium in the Ion Jump Assay Format for Detecting Potassium Channel Openers

The experiment was carried out in accordance with Example 3. However, either 50 μl ES buffer (control) or 50 μl of the KCNQ agonist retigabine (30 μM) were added into defined wells of a plate seeded with cells expressing KCNQ and incubated for 30 min. Then the base line was monitored in the fluorescence reader for 5 min and the fluorescence values were measured for 20 min after the injection of 15 μl of a KCl solution (final concentration 91.8 mM) in each cavity of the plate. The result is depicted in FIG. 4.
FIG. 4A) The time response of the fluorescence intensity of CHO cells expressing KCNQ channels before and after the application (arrow) of 91.8 mM potassium chloride is depicted. The fluorescence responses of the cells in the absence (Δ) and presence (▪) of the known KCNQ channel opener retigabine (30 μM) is shown. The percental change in the fluorescence intensity (ΔF/F) based on the averaged fluorescence intensity before the potassium application is depicted.
FIG. 4B) Difference of the courses of fluorescence in A. The course of fluorescence in the absence of retigabine was subtracted from the course of fluorescence in the presence of retigabine.

### EXAMPLE 5

### Agonistic Activity of Retigabine, Substance A and Substance B in the Ion Jump Assay Format for Detecting Potassium Channel Openers

For this experiment CHO cells (Chinese hamster ovary cells) expressing the KCNQ2 channels and KCNQ3 channels were adherently cultivated in nutrient medium [Minimum Essential Medium (MEM), α medium 22571 1× liquid (Invitrogen), 10% Fetal Calf Serum (FCS) (Gibco, heat inactivated), including selection antibiotics] at 37° C., 5% CO₂ and 95% air humidity in cell culture bottles. At 80% confluency the cells were washed with 1×DPBS without adding Ca²⁺/Mg²⁺ and incubated with 2 ml accutase for 15 min at 37° C. The cells detached from a T75 cell culture bottle were re-suspended in 8 ml nutrient medium. After determining the number of cells in a CASY cell counter, 20,000 cells/well were seeded in Corning® Cell BIND® 96-well plates (black with clear bottom) in 100 μl nutrient medium and incubated for 24 h at 37° C., 5% CO₂ and 95% air humidity. Then the cells were loaded with the red FMP dye of the company Molecular Devices. A vial Membrane Potential Assay Kit Red Component A (MPK, bulk-kit) was dissolved in 200 ml extracellular buffer solution (ES; 120 mM NaCl, 1 mM KCl, 10 mM HEPES, 2 mM CaCl₂, 2 mM MgCl₂, 10 mM glucose; pH 7.4). To load the cells with the voltage-sensitive fluorescent dye, the nutrient medium was discarded first. Then the cells were washed with 200 μl ES buffer; subsequently they were covered with 100 μl of the dye solution and incubated for 45 min. After incubation with the dye, either 50 μl ES buffer (control) or 50 μl of the KCNQ agonist retigabine, substance A or substance B were added into exactly defined wells of the assay plate and incubated for 30 min. The final concentrations in the batch of the KCNQ openers used herein are 0.0625, 0.625, 0.125, 1.25, 2.5, 5 and 10 μM. Then the base line was monitored in the fluorescence reader for 4-5 min, followed by an injection of 15 μl of a KCl solution (final concentration 91.8 mM) in each cavity of the plate and an immediate further measurement in the reader for the following 15 min. The measurements were carried out by the Fluostar or Polarstar fluorescence reader of the company BMG or the FlexStation of the company Molecular Devices. It was excited at a wavelength of 525 nm and detected at a wavelength of 560 nm.

The results are shown in FIGS. 5A+B, 6A+B and 7A+B.
A) The time response of the fluorescence intensity of CHO cells expressing the KCNQ channels before and after the application (arrow) of 91.8 mM potassium chloride is depicted. The fluorescence responses of the cells in the presence of different concentrations of retigabine (FIG. 5A), substance A (FIG. 6A) and substance B (FIG. 7A) are shown. The cells were incubated each with the Membrane Potential Kit (Molecular Devices). The percental change in the fluorescence intensity (ΔF/F) based on the averaged fluorescence intensity before the potassium application is depicted. The course of fluorescence of control cells in which no active agent had been applied was subtracted from the shown courses of fluorescence.
B) The percental changes in the fluorescence intensity (ΔF/F) after the application of 91.8 mM potassium chloride depending on the concentration of retigabine (FIG. 5B), substance A (FIG. 6B) and substance B (FIG. 7B) are depicted. In each case ΔF/F was determined 60 s after the potassium application. Each of the mean values of three measurements is depicted. The ΔF/F for the control cells (in the presence of an active agent) was subtracted. A dose-response curve was adapted to the data points by means of the Hill Equation and, thus, the indicated EC50 values for agonistic activity were determined.

### EXAMPLE 6

### Examination of KCNQ Agonists

The following way of carrying out the experiment describes a preferred protocol by means of which voltage-sensitive potassium channels (exemplified by the KCNQ channels) can be examined in view of channel openers. By means of this protocol all types of potassium channel openers and potassium channel agonists, respectively, can be examined. The aforementioned fluorescent dyes may be used for doing so.

To cultivate the CHO cells (Chinese hamster ovary cells) expressing the KCNQ channels, they are adherently cultivated in Minimum Essential Medium (MEM), a medium 22571 1×liquid (Invitrogen) with 10% Fetal Calf Serum (FCS) (Gibco, heat inactivated) and selection antibiotics at 37° C., 5% CO₂ and 95% air humidity. TC Flask 80 cm² (Nunc) are used as cell culture bottles.

The cells are passaged by decanting the culture medium in a first step and, subsequently, washing the cells with a buffer with a composition of 0.9 mM CaCl₂, 2.7 mM KCl, 1.5 mM KH₂PO₄, 0.5 mM MgCl₂, 140 mM NaCl and 8 mM Na₂HPO₄. To detach the cells from the culture bottle, 2 ml accutase (PAA Laboratories) are added. It is incubated for 15 minutes at 37° C. and, thus, the cells start to roll off (“abkugeln”). The cells are detached from the bottom of the cell culture bottles by slapping with the flat hand at the brim of the culture bottle. Determining the present number of the cells is carried out by a CASY model TCC cell counting device (Schärfe System). The cells are seeded into new culture vessels in 20 ml medium. The following cell numbers are seeded (A=day of seeding the cells, E=day of harvesting the cells): A: Monday—E: Monday—4*10⁵ cells; A: Monday—E: Friday—3*10⁵ cells; A: Thursday—E: Monday—3*10⁵ cells; A: Friday—E: Tuesday—3*10⁵ cells; A: Friday—E: Wednesday—2*10⁵ cells.

For seeding the cells into 96 well measuring plates, the cells are washed and detached from the culture vessel as described above. After determining the present number of cells, 20,000 cells/well are seeded into Corning® CellBIND® 96-well plates (black with clear bottom) in 100 up of the described nutrient medium, incubated for 1 h at room temperature without gassing or regulating the air humidity and for 24 h at 37° C., 5% CO₂ and 95% air humidity.

To be able to load the cells with the used voltage-sensitive dye, a vial Membrane Potential Assay Kit Red Component A (MPK, bulk-kit) is dissolved in 200 ml of extracellular solution (ES; 120 mM NaCl, 1 mM KCl, 10 mM HEPES, 2 mM CaCl₂, 2 mM MgCl₂, 10 mM glucose; pH 7.4). The nutrient medium covering the cells is discarded. The cells are washed with 200 μl ES buffer, subsequently, covered with 100 μl of the dye solution and incubated in the dark for 45 min at room temperature without gassing or regulating the air humidity.

The fluorescence is measured by a Fluostar fluorescence reader (BMG). The fluorescence is excited at 525 nm and detected at 560 nm. After incubation with the dye, the substance to be tested is added in the desired concentration in 50 μl volume or 50 μl ES for control purposes is added and incubated for 30 min. Then the fluorescence intensity of the dye is monitored in the well for 5 min. Subsequently, a second injection of 15 μl of a 100 mM KCl solution (final concentration 91.8 mM) was carried out. The change in the fluorescence is followed for 30 min to obtain all relevant measurement values.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A method for identifying the agonistic activity of a target compound on a potassium channel, said method comprising the steps of:
a) providing a population of cells expressing a potassium channel in a reaction batch and, optionally, a protein-based fluorescent-optical voltage sensor,
b) optionally, incubating the population of cells with a voltage-sensitive fluorescent dye in the reaction batch,
c) adding the target compound to the reaction batch of step a) or step b),
d) determining a value F₁ of the fluorescence intensity of the cells,
e) adding potassium ions in a physiologically acceptable concentration,
f) determining a value F₂ of the fluorescence intensity of the cells, and
g) comparing the fluorescence intensity F₂ with the fluorescence intensity F₁ and determining the agonistic activity of the target compound on the potassium channel.

2. A method according to claim 1 wherein the potassium channel is selected from the group consisting of voltage-dependent potassium channels, inwardly rectifying potassium channels, calcium-active potassium channels and two-pore-domain channels.

3. A method according to claim 2 wherein the potassium channel is a voltage-dependent potassium channel.

4. A method according to claim 3 wherein the voltage-dependent potassium channel is selected from the family of the K_{V} channels.

5. A method according to claim 4 wherein the voltage-dependent potassium channel from the family of the K_{V} channels is a channel of the K_{V}7.x family and KCNQ family.

6. A method according to claim 2 wherein the potassium channel is an inwardly rectifying potassium channel.

7. A method according to claim 6 wherein the inwardly rectifying potassium channel is a K_{ATP} channel.

8. A method according to claim wherein the population of cells expressing the potassium channel is selected from the group consisting of transiently transfected cells, stable cell lines, primary cell cultures, tissue cells and cells endogenously expressing a potassium channel.

9. A method according to claim 1 wherein the fluorescent dye is a voltage-sensitive fluorescent dye.

10. A method according to claim 9 wherein the voltage-sensitive fluorescent dye is selected from the group consisting of slow or fast dyes, FRET-based combinations of dyes, and protein-based fluorescent-optical voltage sensors.

11. A method according to claim 10 wherein the voltage-sensitive fluorescent dye is a fast voltage-sensitive dye selected from the group consisting of styryle dyes, oxonol dyes and hemicyanine dyes.

12. A method according to claim 10 wherein the voltage-sensitive dye is a slow voltage-sensitive fluorescent dye selected from the group consisting of Molecular Devices' Membrane Potential Kit, FRET-based dye systems of the companies Invitrogen or Axiom, carbocyanine dyes, rhodamine dyes, oxonol dyes, bisoxonol dyes and merocyanine dyes.

13. A method according to claim 12, wherein the voltage-sensitive dye is selected from the group consisting of DiSBAC₂(3), DiBAC₄(3), DiOC₂(3), DisC₃(3) and DisC₃(5).

14. A method according to claim 1 wherein the method is configured for use in high-throughput screening.

15. An isolated and purified potassium channel agonist identified by a method comprising the steps of:
a) providing a population of cells expressing a potassium channel in a reaction batch and, optionally, a protein-based fluorescent-optical voltage sensor,
b) optionally, incubating the population of cells with a voltage-sensitive fluorescent dye in the reaction batch,
c) adding the target compound to the reaction batch of step
a) or step b),
d) determining a value F₁ of the fluorescence intensity of the cells,
e) adding potassium ions in a physiologically acceptable concentration,
f) determining a value F₂ of the fluorescence intensity of the cells, and
g) comparing the fluorescence intensity F₂ with the fluorescence intensity F₁ and determining the agonistic activity of the target compound on the potassium channel.

16. A pharmaceutical formulation comprising a potassium channel agonist identified by a method comprising the steps of:
a) providing a population of cells expressing a potassium channel in a reaction batch and, optionally, a protein-based fluorescent-optical voltage sensor,
b) optionally, incubating the population of cells with a voltage-sensitive fluorescent dye in the reaction batch,
c) adding the target compound to the reaction batch of step a) or step b),
d) determining a value F₁ of the fluorescence intensity of the cells,
e) adding potassium ions in a physiologically acceptable concentration,
f) determining a value F₂ of the fluorescence intensity of the cells, and
g) comparing the fluorescence intensity F₂ with the fluorescence intensity F₁ and determining the agonistic activity of the target compound on the potassium channel.

17. A method of treating a disease moderated by at least one potassium channel, said method comprising administering, to a subject in need thereof, a pharmaceutically effective amount of a potassium channel agonist identified by a method comprising the steps of:
a) providing a population of cells expressing a potassium channel in a reaction batch and, optionally, a protein-based fluorescent-optical voltage sensor,
b) optionally, incubating the population of cells with a voltage-sensitive fluorescent dye in the reaction batch,
c) adding the target compound to the reaction batch of step a) or step b),
d) determining a value F₁ of the fluorescence intensity of the cells,
e) adding potassium ions in a physiologically acceptable concentration,
f) determining a value F₂ of the fluorescence intensity of the cells, and
g) comparing the fluorescence intensity F₂ with the fluorescence intensity F₁ and determining the agonistic activity of the target compound on the potassium channel.
